(19) United States
(12) Patent Application Publication
(10) Pub. No.: US 2007/0129589 A1
(43) Pub. Date: Jun. 7, 2007

(54) Metathesis catalyst and process for producing an olefin using the same

(76) Inventors: Masakazu Iwamoto, Kanagawa (JP); Yoshitsugu Kosugi, Shizuoka (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/570,317

(22) PCT Filed: Sep. 3, 2003
(86) PCT No.: PCT/JP03/11265

§ 371 Date: Dec. 1, 2006

(51) Int. Cl. C07C 6/00; B01J 23/00

(52) U.S. Cl. 585/646; 502/300

(57) ABSTRACT

A novel olefin production process for forming at least one olefin from two or more olefins in the presence of a catalyst containing at least one selected from the group consisting of nickel, aluminum, manganese, iron and copper which is supported by a regular mesoporous material. In the above olefin production process, propylene can be formed from ethylene and butene. In the above olefin production process, water vapor can be contained in a reaction gas in a molar ratio based on the total of the raw material olefins of 0.001 to 1.

### TECHNICAL FIELD

The present invention relates to a metathesis catalyst and a process for producing an olefin using the same.

### BACKGROUND ART

A metathesis reaction has been known as means of selectively producing propylene. In this reaction, a heterogeneous catalyst such as a molybdenum-, tungsten-or rhenium-based catalyst supported by silica or a homogeneous catalyst such as a ruthenium or osmium complex has been used. For example, JP-A 62-197147 discloses that a tungsten/silica catalyst which comprises MgO/Al₂ O₃ as a co-catalyst has activity for a metathesis reaction between ethylene and butene.

However, the productions of molybdenum, tungsten, rhenium, ruthenium and osmium all of which are used in the above catalysts are small and their prices are high.

The conversion of ethylene into butene, hexene and propylene by means of a catalyst containing inexpensive nickel supported by a regular mesoporous material is disclosed by Yoshitsugu Kosugi and Masakazu Iwamoto in “Selective Dimerization of Ethylene on Ni-MCM-41”, the collected drafts of the discussion A of the 90^{-th} catalyst panel discussions, p. 138, 4D12.

However, this reaction has low propylene selectivity.

Meanwhile, a method by which a metal is supported by a regular mesoporous material through template ion exchange is disclosed by M. Iwamoto and Y. Tanaka in Catalysis Surveys from Japan, Vol. 5, No.1, p. 25-36 (2001) and by M. Yonemitsu, Y. Tanaka and M. Iwamoto in Chemistry of Materials, Vol. 9, No. 12, p. 2679-2681 (1997). However, the characteristic properties of the above catalyst are still unknown.

Technologies related to supported nickel catalysts are disclosed by Yoshitsugu Kosugi and Masagazu Iwamoto in “Oligomerization of Ethylene on MCM-41”, Lecture Drafts I of the 81-th Spring Annual Convention of the Association of Japanese Chemistry, p. 163, 3C5-16 (2002), by Yoshitsugu Kosugi and Masakazu Iwamoto in “Analysis of Propylene Formation Mechanism of Ethylene Reaction (I) on Ni-MCM-41”, Lecture Drafts I of the 83-th Spring Annual Convention of the Association of Japanese Chemistry, p.183, 1E2-53 (2003) and by Yoshitsugu Kosugi, Takashi Yamamoto and Masakazu Iwamoto in “Direct Synthesis of Propylene from Ethylene by Ni-MCM-41”, Awarded Special Lecture of the 46-th Annual Convention of the 45-th Anniversary of the Foundation of the Society of Petroleum and the 52-th Research Survey, pp. 154-155, D23.

### DISCLOSURE OF THE INVENTION

It is a first object of the present invention to provide a novel metathesis catalyst. It is a second object of the present invention to provide a novel process for producing an olefin.

The metathesis catalyst of the present invention contains at least one selected from the group consisting of nickel, aluminum, manganese, iron and copper which is supported by a regular mesoporous material and forms at least one olefin from two or more olefins.

In the above metathesis catalyst, silica may be used as the main component of a skeleton constituting the regular mesoporous material, and propylene can be formed from ethylene and butene.

In the above metathesis catalyst, a regular mesoporous material having a pore diameter of 2 to 10 nm may be used.

In the above metathesis catalyst, at least one selected from the group consisting of nickel, aluminum, manganese, iron and copper is supported by a regular mesoporous material through template ion exchange.

The process for producing an olefin of the present invention is to form at least one olefin from two or more olefins in the presence of a catalyst containing at least one selected from the group consisting of nickel, aluminum, manganese, iron and copper which is supported by a regular mesoporous material.

In the above process for producing an olefin, propylene can be formed from ethylene and butene.

In the above process for producing an olefin, water vapor can be contained in a reaction gas in a molar ratio based on the total of the raw material olefins of 0.001 to 1.

### BEST MODE FOR CARRYING OUT THE INVENTION

The best mode for carrying out the invention will be described hereinunder.

A description is first given of the metathesis catalyst.

A regular mesoporous material is used as the carrier of the catalyst of the present invention. The regular mesoporous material is an inorganic or inorganic-organic composite solid material having regular nano-pores with a diameter of 2 to 10 nm, particularly preferably a regular mesoporous material comprising silica as the main component of its skeleton. When it has a pore diameter of 2 nm or more, it has advantages that large molecules easily pass therethrough and that a reaction product and a product pass therethrough at a high speed. When it has a pore diameter of 10 nm or less, it has advantages that various active components can be easily supported and that an effect special to the nano-pores is efficiently developed.

Although the method of synthesizing the above regular mesoporous material as a carrier is not particularly limited, a known method in which it is synthesized from a quaternary ammonium salt having a higher alkyl group with 8 or more carbon atoms as a template and a silica precursor may be employed.

As for the type of the silica precursor, colloidal silica, silica gel, alkali silicates such as sodium silicate and potassium silicate, and silicon alkoxides such as tetramethyl orthosilicate and tetraethyl orthosilicate may be used alone or in combination.

Although the template used for the synthesis of the regular mesoporous material is not limited to a particular type, a halogenated alkyltrimethylammonium-based cationic surfactant represented by a general formula CH₃(CH₂)ₙN (CH₃)₃.X (n is an integer of 7 to 21, X is a halogen ion) is particularly preferably used. Specific examples of the alkyltrimethylammonium-based cationic surfactant include n-octyltrimethylammonium bromide, n-decyltrimethylammonium bromide, n-dodecyltrimethylammonium bromide, n-tetradecyltrimethylammonium bromide and n-octadecyltrimethylammonium bromide.

The method of supporting nickel by these carriers is not particularly limited and known methods such as impregnation, vapor deposition and carrier complex decomposition may be employed. Particularly preferred is template ion exchange for exchanging a template ion for a nickel ion in a water medium without baking and removing a template occluded in the pores and used to synthesize the regular mesoporous material.

In the present invention, as for the amount of nickel supported by the regular mesoporous material, the Si/Ni atomic ratio of silicon contained in the silica constituting the skeleton to nickel is preferably 1000 to 5, more preferably 100 to 10.

When the Si/Ni atomic ratio is 5 or more, the formation of nickel oxide fine particles having low catalytic activity can be suppressed. When the Si/Ni atomic ratio is 10 or more, this effect becomes more marked.

When the Si/Ni atomic ratio is 1000 or less, highly dispersed nickel can be fully supported. When the Si/Ni atomic ratio is 100 or less, this effect becomes more marked.

In the present invention, template ion exchange can be carried out by contacting the regular mesoporous material containing the template occluded in its pores to an aqueous solution of an inorganic acid salt or organic acid salt of nickel. Examples of the nickel source include nickel acetate, nickel chloride, nickel bromide, nickel sulfate, nickel oxide, nickel hydroxide and nickel nitrate. These nickel compounds may be used alone or in combination of two or more. Out of these, nickel nitrate is preferred because it is easy to handle and has high solubility in water.

In the present invention, the regular mesoporous material supporting nickel through template ion exchange is preferably heated in an oxygen-existent atmosphere in order to remove the residual template by combustion. This heat treatment is carried out at 200 to 800° C., preferably 300 to 600° C.

When the heat treatment temperature is 200° C. or higher, the combustion of the template is promoted. When the heat treatment temperature is 300° C. or higher, this effect becomes more marked.

When the heat treatment temperature is 800° C. or lower, the disintegration of silica constituting the wall of each pore is prevented. When the heat treatment temperature is 600° C. or lower, this effect becomes more marked.

The metal supported by the regular mesoporous material is not limited to nickel. At least one selected from the group consisting of nickel, aluminum, manganese, iron and copper may be used.

A description is subsequently given of the process for producing an olefin using the metathesis catalyst.

The process for producing an olefin of the present invention is to form at least one olefin from two or more olefins through a metathesis reaction in the presence of a catalyst containing at least one selected from the group consisting of nickel, aluminum, manganese, iron and copper which is supported by a regular mesoporous material.

In the production process of the present invention, it is very important for the highly selective production of propylene that a mixture of ethylene and butene should be used as a raw material used for the reaction. Butene to be mixed with ethylene is 1-butene, cis-2-butene, trans-2-butene or a mixture thereof.

The mixing ratio of ethylene to butene in the raw material gas is generally 1/9 to 9/1, preferably 2/8 to 8/2, more preferably 3/7 to 7/3.

When the mixing ratio is 1/9 or more, the amount of the reacted ethylene increases and the yield of propylene grows. When the mixing ratio is 2/8 or more, this effect becomes more marked. When the mixing ratio is 3/7 or more, this effect becomes much more marked.

When the mixing ratio is 9/1 or less, the amount of the reacted butene increases and the yield of propylene grows. When the mixing ratio is 8/2 or less, this effect becomes more marked. When the mixing ratio is 7/3 or less, this effect becomes much more marked.

The raw material olefins are not limited to the above ethylene and butene. Besides these, linear olefins such as propylene, n-pentene, n-hexene, n-heptene and n-octene, branched olefins such as 1-methyl-2-butene and 1-methyl-2-pentene, and cyclic olefins such as cyclopentene and cyclohexene may be used alone or in combination of two or more.

The raw material gas may contain a saturated hydrocarbon such as methane, ethane or n-butane. The raw material gas is introduced into a reactor directly or after it is diluted with an inert gas such as nitrogen, helium, argon or carbonic acid gas.

In the present invention, to selectively produce propylene, water vapor is preferably existent in a molar ratio based on the total of the raw material olefins of 0.001 to 1. The molar ratio of water vapor to the total of the raw material olefins is more preferably 0.005 to 0.5.

When the molar ratio of water vapor to the total of the raw material olefins is 0.001 or more, it is easy to maintain catalytic activity. When the molar ratio is 0.005 or more, this effect becomes more marked. When the molar ratio based on ethylene is 1 or less, a reduction in catalytic activity which readily occurs in the presence of a surplus of water is prevented. When the molar ratio is 0.5 or less, this effect becomes more marked.

As for the reaction conditions in the production process of the present invention, the reaction temperature is generally 200 to 600° C., preferably 250 to 500° C.

When the reaction temperature is 200° C. or higher, the reaction rate and the reaction activity become high. When the reaction temperature is 250° C. or higher, this effect becomes more marked.

When the reaction temperature is 600° C. or lower, a reduction in the activity of the catalyst is prevented. When the reaction temperature is 500° C. or lower, this effect becomes more marked.

The contact time between the raw material gas and the catalyst in the above reaction is generally 0.01 to 10 g-catalyst-sec/cc-raw material gas, preferably 0.1 to 5 g-catalyst-sec/cc-raw material gas.

When the contact time is 0.01 g-catalyst-sec/cc-raw material gas or more, the conversion of ethylene fully proceeds. When the contact time is 0.1 g-catalyst -sec/cc-raw material gas or more, this effect becomes more marked.

When the contact time is 10 g-catalyst-sec/cc-raw material gas or less, an undesired side reaction is suppressed and the selectivity of propylene can be increased. When the contact time is 5 g-catalyst-sec/cc-raw material gas or less, this effect becomes more marked.

The pressure in the above reaction can be suitably selected from a wide range from normal pressure to high pressure but generally from normal pressure to about 1.0 MPa.

Although the reaction system in the production process of the present invention is not particularly limited, in general, the catalyst is filled into a fixed bed and a circulation system is adopted.

The metathesis reaction is a reaction in which two similar molecules exchange their bonds to form two product molecules having the same (or similar) bonding style.

The present invention can provide a novel metathesis catalyst which contains at least one selected from the group consisting of nickel, aluminum, manganese, iron and copper supported by a regular mesoporous material and forms at least one olefin from two or more olefins.

The present invention can provide a novel process for producing an olefin, which forms at least one olefin from two or more olefins in the presence of a catalyst containing at least one selected from the group consisting of nickel, aluminum, manganese, iron and copper supported by a regular mesoporous material.

It is needless to say that the present invention is not limited to the above best mode for carrying the invention but various changes and modifications may be made in the invention without departing from the spirit and scope thereof.

Examples of the present invention will be described in detail hereinunder. It is needless to say that the present invention is not limited to these examples.

### REFERENCE EXAMPLE 1

(Synthesis of C-12-MCM-41)

225.0 g of n-dodecyltrimethylammonium bromide and 641.4 g of ion exchange water were fed to a 2-liter Teflon (registered trademark) beaker and stirred in a water bath at 40° C. until a homogeneous solution was obtained. A solution prepared by dissolving 11.6 g of sodium hydroxide in 125.5 g of ion exchange water and 306.3 g of colloidal silica (Snowtex of Nissan Chemical Industries, Ltd.) were added to the resulting solution at the same time and stirred for 2 hours. The obtained mixed solution was transferred to an autoclave equipped with a 2-liter Teflon (registered trademark) tube for a thermometer, and the autoclave was closed up so that the solution was left to stand and maintained at 140° C. for 48 hours. After the autoclave was cooled, the contents were taken out and a solid component was separated by suction filtration. After the solid component was rinsed with 2 liters of ion exchange water, it was dried at 80° C. The yield of the obtained solid component was 77.6 g. When the powder X-ray diffraction of this solid component was measured, a diffraction peak was observed at a 2θ of 2.54°, 4.40° and 5.05°. Thus, it was found that the solid component had a regular structure. Further, when this solid component was heated up to 600° C. at a temperature elevation rate of 5° C./min and baked in the air at the same temperature for 6 hours to obtain its pore diameter by a nitrogen adsorption method, it was confirmed that the solid component had an average pore diameter of 2.24 nm.

### EXAMPLE 1

(Preparation of Catalyst)

3 g of unbaked MCM-41 obtained in Reference Example 1 was weighed and fed to a Teflon (registered trademark) vessel. Meanwhile, 0.371 g of nickel nitrate was dissolved in 60 ml of ion exchange water, and the resulting solution was transferred to the Teflon (registered trademark) container of MCM-41 and strongly stirred at room temperature for 1 hour. The Si/Ni atomic ratio of silicon contained in MCM-41 to the added nickel was 40. Thereafter, the vessel was covered with a cellophane film, immersed in a water bath set at 80° C. and left to stand for 20 hours. The solution was cooled to room temperature, and solid matter was separated by suction filtration, rinsed with about 500 ml of ion exchange water and dried at 80° C. The thus obtained solid matter was finely ground, spread thin on a magnetic plate and placed into an electric furnace to be heated up to 600° C. at a temperature elevation rate of 5° C./min and baked for 6 hours. When the thus obtained catalyst was analyzed by an ICP emission spectrum method, its Si/Ni ratio was 46.3.

### EXAMPLE 2

(Reaction Between Ethylene and 1-Butene)

The bottom portion of a quartz glass tube having an inner diameter of 9.80 mm was filled with quartz wool and further with a predetermined amount of the above catalyst. A glass thin tube for inserting a thermocouple for measuring temperature was inserted in the center of the reaction tube and the height of the quartz wool was adjusted to position the end of the thermocouple at the center. The reactor was composed of a mass flow controller, a saturator, a reaction tube and a gas chromatograph for analysis (equipped with a hydrogen flame detector). The flow rate of the reaction gas which was introduced into a gas sampler for the gas chromatograph from above the catalyst and discharged from below the catalyst was controlled by the mass flow controller. The reaction tube was heated in an electric furnace and adjusted to ensure that the temperature of the catalytic layer became a predetermined value. Prior to the reaction, a nitrogen gas was blown at a rate of 50 ml/min at 300° C. for 2 hours as the pretreatment of the catalyst. The reaction was carried out by supplying a mixed gas of ethylene, 1-butene and nitrogen (ethylene content of 4.97 mol %, 1-butene content of 4.97 mol %) at a flow rate of 30 ml/min. The pressure was 0.1 MPa. Water was added by letting the reaction gas pass through the saturator of water cooled to 0° C. (0.6 mol % in the reaction gas).

The results of the reaction which was carried out as described above are shown in Table 1. In the table, hexene is mainly 2-hexene. The term “conversion (%)” means the percent of the number of moles of the reacted raw material component to the number of moles of the raw material component before the reaction. The “yield (C-mol %)” is obtained by the expression ((number of moles of formed component × number of carbon atoms of formed component)/(number of moles of raw material component before reaction × number of carbon atoms of raw material component)) × 100. The same shall apply to Table 2 and Table 3 below.

TABLE 1

| | amount of | reaction | conversion of | conversion of | yield of | yield of |
|---|---|---|---|---|---|---|
| Experiment | catalyst | temperature | ethylene | 1-butene | propylene | hexene |
| No. | (g) | (° C.) | (%) | (%) | (C-mol %) | (C-mol %) |
| 1 | 0.30 | 200 | 0.3 | 2.9 | 0.1 | 0.0 |
| 2 | 0.30 | 250 | 7.3 | 0.1 | 0.6 | 0.0 |
| 3 | 0.30 | 300 | 16.2 | 0.3 | 2.5 | 0.1 |
| 4 | 0.30 | 350 | 23.2 | 8.5 | 8.7 | 0.5 |
| 5 | 0.30 | 400 | 31.1 | 10.7 | 11.4 | 0.0 |
| 6 | 0.30 | 450 | 22.9 | 24.0 | 17.6 | 0.1 |
| 7 | 0.10 | 350 | 9.2 | 1.8 | 2.7 | 0.0 |
| 8 | 0.10 | 400 | 7.7 | 7.1 | 3.8 | 0.0 |
| 9 | 0.10 | 450 | 5.9 | 4.9 | 6.2 | 0.0 |

It is understood from Table 1 that propylene can be selectively formed by reacting a mixed gas of ethylene and 1 butene in the presence of a nickel catalyst supported by a regular mesoporous material.

In this reaction, 1-butene was first isomerized on the acid site of the catalyst and converted into 2-butene. It is considered that a metathesis reaction proceeded between ethylene and 2-butene thereafter.

### EXAMPLE 3

(Reaction Between Ethylene and Trans-2-Buetne)

The procedure of Example 2 was repeated except that the raw material gas was changed from the mixed gas of ethylene, 1 butene and nitrogen to a mixed gas of ethylene, trans-2-butene and nitrogen (ethylene content of 4.97 mol %, trans-2-butene content of 4.97 mol %). The results of the reaction are shown in Table 2.

TABLE 2

| | amount of | reaction | conversion of | conversion of | yield of | yield of |
|---|---|---|---|---|---|---|
| Experiment | catalyst | temperature | ethylene | 2-butene | propylene | hexene |
| No. | (g) | (° C.) | (%) | (%) | (C-mol %) | (C-mol %) |
| 10 | 0.30 | 200 | 0.0 | 1.8 | 0.0 | 0.0 |
| 11 | 0.30 | 250 | 4.8 | 0.6 | 0.1 | 0.0 |
| 12 | 0.30 | 300 | 0.0 | 3.0 | 0.3 | 0.0 |
| 13 | 0.30 | 350 | 26.7 | 4.5 | 2.5 | 0.1 |
| 14 | 0.30 | 400 | 37.1 | 3.9 | 10.1 | 0.0 |
| 15 | 0.30 | 450 | 20.2 | 21.8 | 16.0 | 0.0 |

It is understood from Table 2 that propylene can be selectively formed by reacting a mixed gas of ethylene and trans-2-butene in the presence of a nickel catalyst supported by a regular mesoporous material.

It is considered that metathesis proceeds directly on the active site in this reaction.

### EXAMPLE 4

(Reaction of Propylene)

The procedure of Example 2 was repeated except that the raw material gas was changed from the mixed gas of ethylene, 1-butene and nitrogen to a mixed gas of propylene and nitrogen (propylene content of 9.94 mol %). The results of the reaction are shown in Table 3.

TABLE 3

| | amount of | reaction | conversion of | yield of | yield of | yield of |
|---|---|---|---|---|---|---|
| Experiment | catalyst | temperature | propylene | ethylene | butene | hexene |
| No. | (g) | (° C.) | (%) | (C-mol %) | (C-mol %) | (C-mol %) |
| 16 | 0.30 | 200 | 2.3 | 0.0 | 0.0 | 0.9 |
| 17 | 0.30 | 250 | 6.0 | 0.1 | 0.4 | 1.7 |
| 18 | 0.30 | 300 | 11.6 | 1.0 | 2.7 | 2.1 |
| 19 | 0.30 | 350 | 17.1 | 3.1 | 7.1 | 1.4 |
| 20 | 0.30 | 400 | 26.2 | 5.3 | 12.3 | 1.5 |
| 21 | 0.30 | 450 | 24.5 | 8.9 | 17.3 | 0.2 |

It is known that a reverse reaction proceeds on a metathesis reaction catalyst. It could be confirmed from this example that a reverse reaction actually proceeds efficiently on the catalyst of the present invention.

1. A metathesis catalyst containing at least one selected from the group consisting of nickel, aluminum, manganese, iron and copper which is supported by a regular mesoporous material, wherein
at least one olefin is formed from two or more olefins.

2. The metathesis catalyst according to claim 1, wherein the raw material olefins are ethylene and butene, the formed olefin is propylene, and the main component of a skeleton constituting the regular mesoporous material is silica.

3. The metathesis catalyst according to claim 2, wherein a regular mesoporous material having a pore diameter of 2 to 10 nm is used.

4. The metathesis catalyst according to claim 1, wherein at least one selected from the group consisting of nickel, aluminum, manganese, iron and copper is supported by a regular mesoporous material through template ion exchange.

5. A process for producing an olefin, comprising forming at least one olefin from two or more olefins in the presence of a catalyst containing at least one selected from the group consisting of nickel, aluminum, manganese, iron and copper which is supported by a regular mesoporous material.

6. The process for producing an olefin according to claim 5, wherein the raw material olefins are ethylene and butene, and the formed olefin is propylene.

7. The process for producing an olefin according to claim 5, wherein water vapor is contained in a reaction gas in a molar ratio based on the total of the raw material olefins of 0.001 to 1.
